(19) United States
(12) Patent Application Publication
(10) Pub. No.: US 2021/0062152 A1
(43) Pub. Date: Mar. 4, 2021

(54) THREE-DIMENSIONAL CELL SPHEROID WITH HIGH PROLIFERATION ACTIVITY, AND PRODUCING METHOD AND USE THEREFOR

(71) Applicant: METATECH (AP) INC., NEW TAIPEI CITY (TW)

(72) Inventors: Tsung-Chi CHEN, NEW TAIPEI CITY (TW); Chih-Hui YANG, NEW TAIPEI CITY (TW); Hen-Yu LIU, NEW TAIPEI CITY (TW); Yen-Chun CHEN, NEW TAIPEI CITY (TW); Chiao-Hsuan TING, NEW TAIPEI CITY (TW)

(21) Appl. No.: 17/001,817

(22) Filed: Aug. 25, 2020

(30) Foreign Application Priority Data
Sep. 4, 2019 (TW) 108131893

(51) Int. Cl. C12N 5/077; A61K 35/33

(52) U.S. Cl. CPC C12N 5/0656; C12N 2535/00; C12N 2513/00; A61K 35/33

(57) ABSTRACT

The present invention discloses a three-dimensional cell spheroid with high proliferation activity, and the cell spheroid is obtained by ejecting a cell aggregate through a needle with a needle gauge of less than 30G. The present invention further provides the producing method and the use for the cell spheroid.

### CROSS REFERENCE

This non-provisional application claims priority of Taiwan Invention Patent Application No. 108131893, filed on Sep. 4, 2019, the contents thereof are incorporated by reference herein.

### FIELD OF THE INVENTION

The present invention is related to a cell spheroid, and its producing method and use, and more particularly to a three-dimensional cell spheroid with high proliferation activity, and its producing method and use.

### BACKGROUND OF THE INVENTION

In the traditional cell therapy, single cells are obtained from a culture dish using enzyme treatment, then is filled into a syringe, and finally injected into a body through the needle of the syringe. However, enzyme can damage the extracellular matrix that contributes several normal cell functions so as to affect the proliferation activity and the biological factor expression level of the cell after injection into the body.

Accordingly, there is a need to improve the traditional cell therapy.

### SUMMARY OF THE INVENTION

The present invention is made based on the unexpected discovery that a cell aggregate formed of multiple cells has increased cell proliferation activity and increased biological factor expression level after ejection through a needle with a specific inner diameter size. Additionally, the thus-obtained cell spheroid has low carcinogenesis risk.

Therefore, an objective of the present invention is to provide a three-dimensional cell spheroid, which is obtained by ejecting a cell aggregate through a needle with a needle gauge of less than 30 G.

Preferably, the cell aggregate contains 30-3,000 cells. Preferably, the cell aggregate is in the form of a regular three-dimensional shape, an irregular three-dimensional shape, a regular spherical shape, or an irregular spherical shape.

Preferably, the cell aggregate is obtained by incubating dermal cells, vascular endothelial cells, fibroblasts, adipocytes, epidermal cells, epithelial cells, mammary glandular cells, muscle cells, islet cells, corneal cells, hair follicle cells, chondrocytes, osteocytes, nerve cells, lung cells, periodontal ligament cells, T cells, B cells, monocytes, macrophages, granulocytes, mast cells, antigen-presenting cells, peripheral blood stem cells, adipose tissue-derived stem cells, or bone marrow mesenchymal stem cells.

Preferably, the needle gauge is of less than 27 G.

Preferably, the needle gauge is of 27G-21 G.

According to the present invention, the three-dimensional cell spheroid has the properties of high proliferation activity and high expression level of specific biological factors, and further has low carcinogenesis risk. As such, the three-dimensional cell spheroid can rapidly grow and retain its high biological activity after implanted into a subject. Since the procedure of cell aggregate ejection through a needle corresponds to the injection procedure for medical treatment or beauty treatment, the three-dimensional cell spheroid implantation into a subject can be performed by means of the cell aggregate ejection through a needle, or the three-dimensional cell spheroid is implanted into a subject, e.g. through injection, after it is incubated in vitro.

According to the foregoing characteristics, the present invention also provides a method for disease treatment or beauty treatment, the method including: implanting a bio-agent comprising the foregoing three-dimensional cell spheroid into a subject in need thereof.

Preferably, the disease treatment is treatment for solid cancer, hematologic malignancy, lower extremity peripheral arterial disease, skin wound, subcutaneous tissue defect, soft tissue defect, degenerative joint disease, knee cartilage defect, stroke, or spinal cord injury.

Preferably, the beauty treatment is treatment for wrinkle elimination, skin pit filling, skin scar filling, soft tissue augmentation, diabetic wound healing, bum wound healing, cut wound healing, or surgical wound healing.

Within the scope of the present invention, a method for producing a three-dimensional cell spheroid is provided, which includes: incubating cells to form a cell aggregate; and ejecting the cell aggregate through a needle with a needle gauge of less than 30 G.

### BRIEF DESCRIPTION OF THE DRAWINGS

FIG. 1 is a schematic drawing illustrating the formation of a cell aggregate according to an embodiment of the present invention;

FIG. 2 is a bar graph illustrating the proliferation activity of three-dimensional cell spheroids obtained using different needle gauges according to Experimental Example and Comparative Example;

FIG. 3 is a bar graph illustrating the biological factor expression level of three-dimensional cell spheroids obtained using 27 G needles according to Experimental Example and Comparative Example; and

FIG. 4 is a bar graph illustrating the sternness-related factor expression level of three-dimensional cell spheroids obtained using 27 G needles according to Experimental Example and Comparative Example.

### DETAILED DESCRIPTION OF THE INVENTION

The detailed description and preferred embodiments of the invention will be set forth in the following content, and provided for people skilled in the art to understand the characteristics of the invention.

An embodiment of the present invention discloses a method for producing a three-dimensional cell spheroid. Since the produced cell spheroid has the properties of high proliferation activity, high expression level of specific biological factors, and low carcinogenesis risk, it can be implanted into a subject for the purpose of disease treatment or beauty treatment. The production method comprises the steps of: incubating cells to form a cell aggregate; and ejecting the cell aggregate through a needle with a needle gauge of less than 30G.

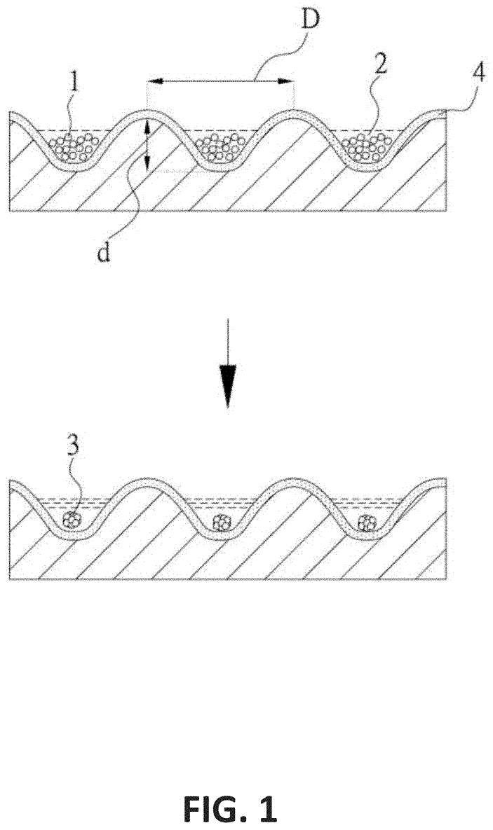

As shown in FIG. 1, the cell aggregate formation is exemplarily illustrated, but not limited thereto. First, the cells (1) are seeded into a recess (2), and an example of the cells is, but not limited to, dermal cells, vascular endothelial cells, fibroblasts, adipocytes, epidermal cells, epithelial cells, mammary glandular cells, muscle cells, islet cells, corneal cells, hair follicle cells, chondrocytes, osteocytes, nerve cells, lung cells, periodontal ligament cells, T cells, B cells, monocytes, macrophages, granulocytes, mast cells, antigen-presenting cells, peripheral blood stem cells, adipose tissue-derived stem cells, or bone marrow mesenchymal stem cells. Next, the cells (1) are incubated to form a cell aggregate (3). It is noted that while the cells (1) are anchorage-dependent cells, the cells (1) can naturally cluster to form the cell aggregate (3) after incubation; while the cells are suspension cells, the cells (1) can cluster to form the cell aggregate (3) through centrifugation after incubation. Additionally, the cell aggregate (3) may be in the form of, but not limited to, a regular three-dimensional shape, an irregular three-dimensional shape, a regular spherical shape, or an irregular spherical shape, and it may contain, but not limited to, 30-3,000 cells. Further, while the cell aggregate (3) is in the form of a regular spherical shape or an irregular spherical shape, the recess (2) may be coated with an adhesion-reducing layer (4), and an example of the adhesion-reducing material is, but not limited to, 2-methacryloyloxyethyl phosphorylcholine polymer. The adhesion-reducing layer (4) can help the cells (1) piled up to form the cell aggregate (3). While the cell aggregate (3) is in the form of a regular three-dimensional shape or an irregular three-dimensional shape, the recess (2) may be coated with an external stimuli-responsive layer (4), and an example of the external stimuli-responsive material is, but not limited to, a photo-responsive material, a pH-responsive material, an electro-responsive material, a magnetic-responsive material, or a chemo-responsive material. The external stimuli can change the hydrophilic-hydrophobic property of the external stimuli-responsive material so that the cell aggregate (3) can be detached from the surface of the recess (2). That is, the adhesion-reducing material and the external stimuli-responsive material both can prevent the adhesion proteins between cells from being damaged so that the structure and the biological activity of the later-obtained cell spheroid can be retained. Additionally, the recess (2) size can control the size and the cell number of the cell aggregate (3). For example, its depth (d) is, but not limited to, 100 μm-400 μm, and its diameter (D) is, but not limited to, 200 μm-1,000 μm.

The foregoing needle gauge is defined based on the Birmingham gauge system. For example, “a needle gauge of 30 G” indicates that a needle has an outer diameter of 0.3112 mm and an inner diameter of 0.15 mm; “a needle gauge of 2 7G” indicates that a needle has an outer diameter of 0.4128 mm and an inner diameter of 0.21 mm; “a needle gauge of 21 G” indicates that a needle has an outer diameter of 0.8192 mm and an inner diameter of 0.514 mm. Preferably, the needle gauge is of less than 27 G, and more preferably, is of 27 G-21 G.

Another embodiment of the present invention is made based on the unexpected discovery that the foregoing three-dimensional cell spheroid can rapidly proliferate in vivo and have the biological activity. Specifically, a method for disease treatment or beauty treatment is disclosed, which includes the step(s) of: implanting a bio-agent comprising the foregoing three-dimensional cell spheroid into a subject in need thereof. The bio-agent can be implanted into the subject by means of that a cell aggregate is ejected through a needle or can be implanted into the subject after the three-dimensional cell spheroid is incubated in vitro so that the purpose of disease treatment or beauty treatment is achieved. Said “disease treatment” indicates, but not limited to, the treatment for solid cancer, hematologic malignancy, lower extremity peripheral arterial disease, skin wound, subcutaneous tissue defect, soft tissue defect, degenerative joint disease, knee cartilage defect, stroke, or spinal cord injury; said “beauty treatment” indicates, but not limited to, the treatment for wrinkle elimination, skin pit filling, skin scar filling, soft tissue augmentation, diabetic wound healing, burn wound healing, cut wound healing, or surgical wound healing.

### Experimental Example

Human fibroblasts were seeded into a culture dish coated with an external stimuli-responsive layer and incubated in Fibroblast Growth Medium (116-500) under a normal condition (37° C. and 5% CO₂). After the cells clustered to form cell aggregates each containing 30-3,000 cells, the culture medium was replaced with a fresh culture one and the culture dish was placed under the corresponding external stimuli so that the formed cell aggregates floated on the culture medium. Afterwards, the cell aggregates were filled into a syringe having a needle with a specific needle gauge. The plunger of the syringe was pushed along the inner of the tube so that the cell aggregates were ejected through the needle to seed into another culture dish. Finally, the thus-obtained three-dimensional cell spheroids were incubated in Fibroblast Growth Medium (116-500) under a normal condition (37° C. and 5% CO₂).

### Comparative Example

Human fibroblasts were seeded into a culture dish and incubated in Fibroblast Growth Medium (116-500) under a normal condition (37° C. and 5% CO₂). After the cell density reached the specific range, the culture medium was removed and the cells were washed with a PBS buffer. Afterwards, 0.25% trypsin was added into the culture dish and incubated under 37° C. for 3 minutes to detach the cells. A fresh culture medium was added into the culture dish to stop the enzymatic reaction and then the cells were resuspended to obtain a single-cell suspension. The single cells were filled into a syringe having a needle with a specific needle gauge. The plunger of the syringe was pushed along the inner of the tube so that the single cells were ejected through the needle to seed into another culture dish. Finally, the thus-obtained cell spheroids were incubated in Fibroblast Growth Medium (116-500) under a normal condition (37° C. and 5% CO₂), wherein the cell number of the thus-obtained cell spheroids was the same as that of the three-dimensional cell spheroids obtained in Experimental Example.

### Analysis Example 1

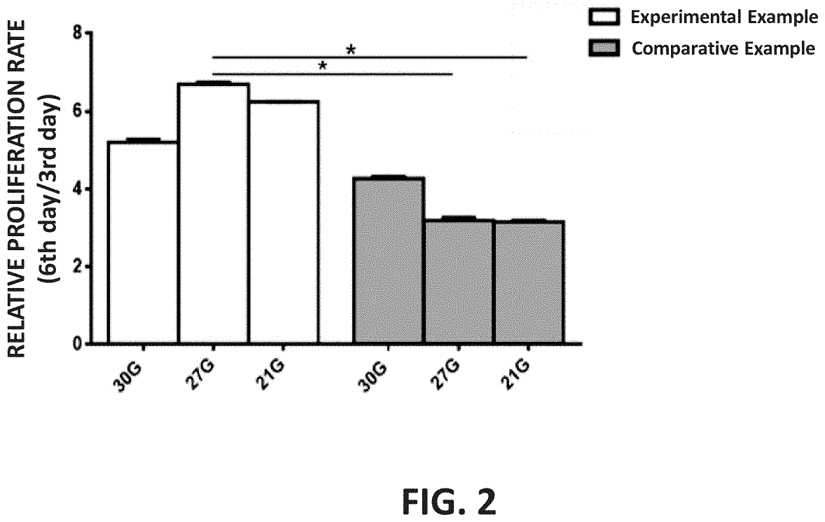

As described in J Pharm Pharmacol. 2015 May;67(5):640-50, since fibroblasts were recovered on the 3rd day after the needle ejection, the relative proliferation rate of cell spheroids obtained in Experimental Example or Comparative Example was calculated based the proliferation rate of the corresponding cell spheroids at the 3rd day after the needle ejection (the cell spheroid incubation). As shown in FIG. 2, while the cell spheroids obtained in Comparative Example were obtained using a needle with a relatively large inner diameter, the relative proliferation rate thereof was relatively low. However, while the cell spheroids obtained in Experimental Example were obtained using a 30 G needle, the relative proliferation rate thereof was the lowest; while the cell spheroids obtained in Experimental Example were obtained using a 21 G needle, the relative proliferation rate thereof was the second lowest; while the cell spheroids obtained in Experimental Example were obtained using a 27 G needle, the relative proliferation rate thereof was the highest.

### Analysis Example 2

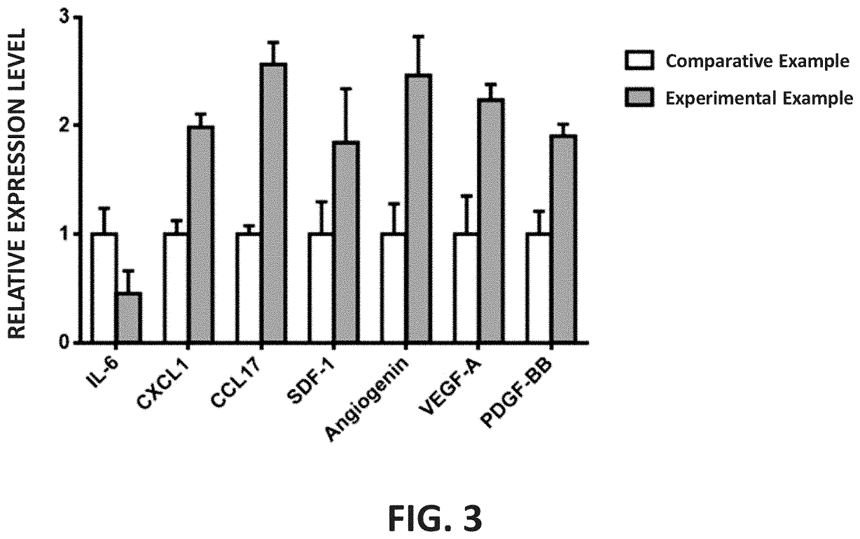

After the foregoing cell spheroids were incubated for 3 days, they were washed with a PBS buffer, and then incubated in a serum-free culture medium under a normal condition (37° C. and 5% CO₂) for 24 hours. Finally, the supernatant was collected for the cytokine array analysis to compare the growth factor expression level of the cell spheroids obtained in Experimental Example with that of the cell spheroids obtained in Comparative Example. As shown in FIG. 3, under the condition of being obtained using a 27 G needle, the expression levels of CXCL-1 ((C-X-C motif) ligand-1), CCL-17 (chemokine (C-C motif) ligand-17), SDF-1 (stromal cell-derived factor-1), angiogenin, VEGF-A (vascular endothelial growth factor-A, and PDGF-BB (platelet-derived growth factor-BB) of the cell spheroids obtained in Experimental Example were higher than those of the cell spheroids obtained in Comparative Example. As known in prior art, CCL-17 can promote the fibroblast migration, SDF-1 can promote the keratinocyte proliferation, angiogenin and VEGF-A both can lead to angiogenesis, and PDGF-BB can promote the cell proliferation; all those factors are related to wound healing. As further shown in FIG. 3, the expression level of IL-6 (interleukin-6) of the cell spheroids obtained in Experimental Example was lower than that of the cell spheroids obtained in Comparative Example, which implied that the cell spheroids obtained in Comparative Example were suffered from damage due to the release the relatively high amount of proinflammatory factors.

### Analysis Example 3

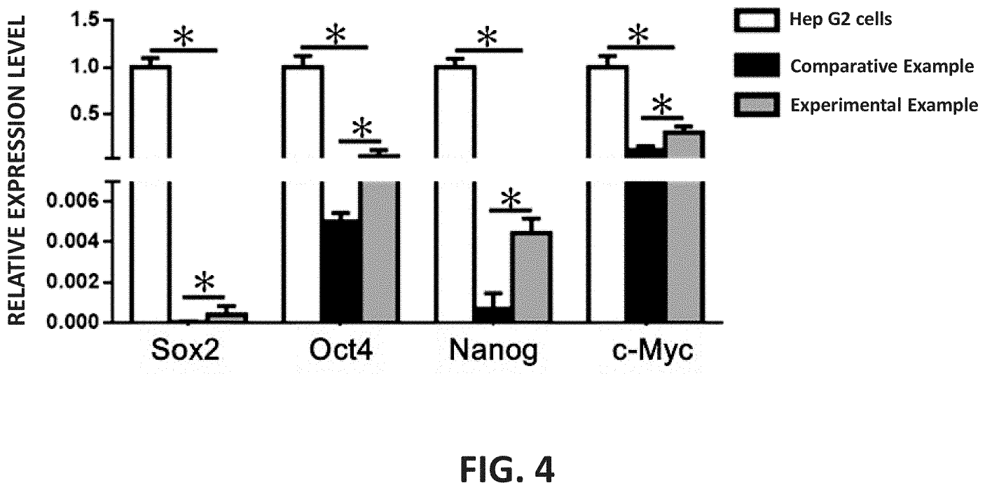

After the foregoing cell spheroids were incubated for 3 days, they were collected for q-PCR (quantitative PCR) to compare the stemness factor expression level of the cell spheroids obtained in Experimental Example with that of the cell spheroids obtained in Comparative Example. As shown in FIG. 4, under the condition of being obtained using a 27G needle, the expression levels of Sox2 (Sex-determining Region Y (SRY)-related Box 2), Oct4(octamer-binding transcription factor 4), Nanog, and c-Myc of the cell spheroids obtained in Experimental Example were higher than those of the cell spheroids obtained in Comparative Example, but lower than those of hepatocellular carcinoma cells (Hep G2 cells). This implied that the cell spheroids obtained in Experimental Example had low carcinogenesis risk.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A three-dimensional cell spheroid, being produced by a method comprising:
seeding cells into a recess;
incubating the cells in the recess to form a cell aggregate; and
ejecting the cell aggregate through a needle with a needle gauge of less than 30 G.

2. The cell spheroid as claimed in claim 1, wherein the cells are selected from the group consisting of: dermal cells, vascular endothelial cells, fibroblasts, adipocytes, epidermal cells, epithelial cells, mammary glandular cells, muscle cells, islet cells, corneal cells, hair follicle cells, chondrocytes, osteocytes, nerve cells, lung cells, periodontal ligament cells, T cells, B cells, monocytes, macrophages, granulocytes, mast cells, antigen-presenting cells, peripheral blood stem cells, adipose tissue-derived stem cells, and bone marrow mesenchymal stem cells, and the cell aggregate contains 30-3,000 cells.

3. The cell spheroid as claimed in claim 2, wherein the cells are fibroblasts, and the recess is coated with an adhesion-reducing layer or an external stimuli-responsive layer.

4. The cell spheroid as claimed in claim 2, wherein the cells are fibroblasts, the recess is coated with an adhesion-reducing layer or an external stimuli-responsive layer, and the recess has a depth of 100 μm-400 μm and a diameter of 200 μm-1,000 μm.

5. The cell spheroid as claimed in claim 4, wherein the needle gauge is of 27 G-21 G.

6. The cell spheroid as claimed in claim 4, wherein the needle gauge is of 30 G-27 G.

7. A method for producing a three-dimensional cell spheroid, comprising:
seeding cells into a recess;
incubating the cells in the recess to form a cell aggregate; and
ejecting the cell aggregate through a needle with a needle gauge of less than 30 G.

8. The method as claimed in claim 7, wherein the cells are selected from the group consisting of: dermal cells, vascular endothelial cells, fibroblasts, adipocytes, epidermal cells, epithelial cells, mammary glandular cells, muscle cells, islet cells, corneal cells, hair follicle cells, chondrocytes, osteocytes, nerve cells, lung cells, periodontal ligament cells, T cells, B cells, monocytes, macrophages, granulocytes, mast cells, antigen-presenting cells, peripheral blood stem cells, adipose tissue-derived stem cells, and bone marrow mesenchymal stem cells, and the cell aggregate contains 30-3,000 cells.

9. The method as claimed in claim 7, wherein the cells are fibroblasts, and the recess is coated with an adhesion-reducing layer or an external stimuli-responsive layer.

10. The method as claimed in claim 7, wherein the cells are fibroblasts, the recess is coated with an adhesion-reducing layer or an external stimuli-responsive layer, and the recess has a depth of 100 μm-400 μm and a diameter of 200 μm-1,000 μm.

11. The method as claimed in claim 10, wherein the needle gauge is of 27 G-21 G.

12. The method as claimed in claim 10, wherein the needle gauge is of 30 G-27 G.

13. A method for disease treatment or beauty treatment, comprising:
implanting a bio-agent comprising a three-dimensional cell spheroid into a subject in need thereof, wherein the three-dimensional cell spheroid is produced by a method comprising:
seeding cells into a recess;
incubating the cells in the recess to form a cell aggregate; and
ejecting the cell aggregate through a needle with a needle gauge of less than 30 G.

14. The method as claimed in claim 13, the disease treatment is treatment for solid cancer, hematologic malignancy, lower extremity peripheral arterial disease, skin wound, subcutaneous tissue defect, soft tissue defect, degenerative joint disease, knee cartilage defect, stroke, or spinal cord injury.

15. The method as claimed in claim 13, the beauty treatment is treatment for wrinkle elimination, skin pit filling, skin scar filling, soft tissue augmentation, diabetic wound healing, burn wound healing, cut wound healing, or surgical wound healing.

16. The method as claimed in claim 13, wherein the cells are selected from the group consisting of: dermal cells, vascular endothelial cells, fibroblasts, adipocytes, epidermal cells, epithelial cells, mammary glandular cells, muscle cells, islet cells, corneal cells, hair follicle cells, chondrocytes, osteocytes, nerve cells, lung cells, periodontal ligament cells, T cells, B cells, monocytes, macrophages, granulocytes, mast cells, antigen-presenting cells, peripheral blood stem cells, adipose tissue-derived stem cells, and bone marrow mesenchymal stem cells, and the cell aggregate contains 30-3,000 cells.

17. The method as claimed in claim 15, wherein the cells are fibroblasts, and the recess is coated with an adhesion-reducing layer or an external stimuli-responsive layer.

18. The method as claimed in claim 15, wherein the cells are fibroblasts, the recess is coated with an adhesion-reducing layer or an external stimuli-responsive layer, and the recess has a depth of 100 μm-400 μm and a diameter of 200 μm-1,000 μm.

19. The method as claimed in claim 18, wherein the needle gauge is of 27 G-21 G.

20. The method as claimed in claim 18, wherein the needle gauge is of 30 G-27 G.
